(19) United States
(12) Patent Application Publication
(10) Pub. No.: US 2014/0378816 A1
(43) Pub. Date: Dec. 25, 2014

(54) INFORMATION PROVIDING METHOD AND MEDICAL DIAGNOSIS APPARATUS FOR PROVIDING INFORMATION

(71) Applicant: Samsung Electronics Co., Ltd., Gyeonggi-do (KR)

(72) Inventors: Keum-yong OH, Gyeonggi-do (KR); Hei-soog KIM, Gyeonggi-do (KR); Dae-hyun BAN, Seoul (KR); Sung-pil JUNG, Gyeonggi-do (KR); Jae-moon JO, Gyeonggi-do (KR); Byung-sun CHOI, Gyeonggi-do (KR); Yang-lim CHOI, Gyeonggi-do (KR)

(21) Appl. No.: 14/310,562

(22) Filed: Jun. 20, 2014

(30) Foreign Application Priority Data
Jun. 21, 2013 (KR) 10-2013-0071947

(51) Int. Cl. A61B 5/00; A61B 5/055; A61B 6/03; A61B 5/11

(52) U.S. Cl. CPC A61B 5/7282; A61B 5/1128; A61B 5/0059; A61B 5/0555; A61B 6/032; A61B 5/7405; A61B 5/742
USPC 600/409; 600/595; 600/407; 600/476; 600/410; 600/425

(57) ABSTRACT

A method of providing medical imaging guidance information for use in operating a medical imaging apparatus detects motion of an examinee placed on a support table positioned for imaging by a medical imaging device. The method compares a level of the detected motion with a predetermined threshold value and adaptively selects and provides guidance information enabling reducing impairment of image quality due to examinee physical motion, in response to the comparison.

### CLAIM OF PRIORITY

This application claims the benefit of Korean Patent Application No. 10-2013-0071947, filed on Jun. 21, 2013 in the Korean Intellectual Property Office, the disclosure of which is incorporated herein its entirety by reference.

### BACKGROUND

1. Field of the Invention

A medical imaging system is used to provide information regarding motion of an examinee such as a patient.

2. Description of the Related Art

A computer tomography (CT) system captures a plurality of X-ray images while the CT system rotates around one or more axes with respect to an object, and synthesizes the plurality of X-ray images to provide a 3D (three dimensional) image volume. Since the CT system is capable of providing a cross-sectional image of the object, the CT system may express an inner structure of the object without image overlaps.

Magnetic resonance imaging (MRI) involves imaging information that is obtained by exposing nuclei to a magnetic field and resonating the nuclei. An MRI apparatus is advantageous in that it is noninvasive, exhibits an excellent soft tissue contrast, compared to a CT apparatus, and the MRI apparatus does not generate artifacts due to bone tissue. Also, the MRI apparatus captures various cross-sectional images in particular directions without moving an object.

A motion of an examinee (e.g., a human patient), which occurs while the examinee is being diagnosed, forms a motion artifact in the MRI image. The motion artifact may be shown as bright noise in the MRI image or as an unnecessary shape having repeating densities. In particular, the motion artifact frequently occurs while capturing an image of a patient who repeatedly moves or while capturing an image of a child.

### SUMMARY

A system detects and minimizes a motion artifact of an examinee being imaged by a medical imaging system for use in controlling a diagnosis process and a medical imaging apparatus.

A method of providing medical imaging guidance information for use in operating a medical imaging apparatus detects motion of an examinee placed on a support table positioned for imaging by a medical imaging device. The method compares a level of the detected motion with a predetermined threshold value and adaptively selects and provides guidance information enabling reducing impairment of image quality due to examinee physical motion, in response to the comparison.

In a feature, the guidance information comprises information regarding the level of the detected motion and information regarding the threshold value, provided by using at least one of text data, graphical data, and audio data. Further, if the level of the detected motion is equal to or greater than the threshold value, the guidance information comprises information indicating that recapture of an image of the examinee is necessary.

In another feature, a medical imaging apparatus comprises a sensor, a motion calculation unit and an output unit. The sensor detects motion of an examinee placed on a support table positioned for medical imaging. The motion calculation unit compares a level of the detected motion with a predetermined threshold value. An output unit provides guidance information that is used to guide the examinee relating to the motion in response to the comparison.

### BRIEF DESCRIPTION OF THE DRAWINGS

The above and other features and advantages of the present invention will become more apparent by describing in detail exemplary embodiments thereof with reference to the attached drawings in which:

FIG. 1 shows a medical imaging apparatus according to invention principles;

FIG. 2 shows a structure of a medical imaging apparatus according to invention principles;

FIG. 3 shows a flowchart of a method of providing information, according to invention principles;

FIG. 4 shows a flowchart of an information providing method, according to invention principles;

FIG. 5 shows a flowchart of an information providing method, according to invention principles;

FIG. 6 shows a flowchart of an information providing method, according to invention principles;

FIG. 7 shows a flowchart of an information providing method, according to invention principles;

FIG. 8 shows a method of providing guide information in response to level of detected motion, according to invention principles;

FIG. 9 shows use of a threshold value of a motion, according to invention principles;

FIG. 10 shows a method of providing guide information, according to invention principles;

FIG. 11A, FIG. 11B, AND FIG. 11C illustrate detection of motion of an examinee using cameras, according to invention principles;

FIG. 12 shows a system for detecting motion of an examinee using a camera, according to invention principles;

FIG. 13A and FIG. 13B show detection of motion of examinees using different types of cameras, according to invention principles;

FIG. 14 is a diagram showing presentation of guide information concerning motion, according to invention principles;

FIG. 15A and FIG. 15B show a system of providing guide information through in-bore displays, according to invention principles;

FIG. 16 shows a system for providing an examinee with guide information, according to invention principles;

FIG. 17 shows presentation of guide information by using an image of an examinee, according to invention principles;

FIG. 18 shows presentation of a menu indicating motion detection, according to invention principles;

FIG. 19A and FIG. 19B show a system for resuming suspended protocols, according to invention principles; and

FIG. 20 shows presentation of displayed medical images indicating motion detection, according to invention principles.

### DETAILED DESCRIPTION

Terms including descriptive or technical terms which are used herein should be construed as having meanings that are obvious to one of ordinary skill in the art. However, the terms may have different meanings according to an intention of one of ordinary skill in the art, precedent cases, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

Also, when a part “includes” or “comprises” an element, unless there is a particular description contrary thereto, the part can further include other elements, not excluding the other elements. In the following description, terms such as “unit” may be embodied, but is not limited to, as software or a hardware component, such as a field programmable gate array (FPGA) or an application specific integrated circuit (ASIC). However, a unit may advantageously be configured to reside on the addressable storage medium and configured to execute on one or more processors. Thus, a unit may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and units may be combined into fewer components and units or further separated into additional components and units.

Throughout the specification, an “image” may mean multi-dimensional data including discrete image elements (e.g., pixels of a two-dimensional (2D) image and voxels of a three-dimensional (3D) image). For example, the image may include a medical image of an object which is obtained by using an X-ray, computer tomography (CT), magnetic resonance imaging (MRI), an ultrasonic wave, or other medical diagnosis systems.

Also, throughout the specification, an “object” may include human, animal, or a part of human or animal. For example, the object may include organs such as a liver, heart, womb, brain, breast, abdomen, or the like, or a blood vessel. Also, the object may include a phantom. The phantom means a material having a volume that is very close to a density and effective atomic number of an organism, and may include a sphere phantom having a characteristic similar to a physical body.

Throughout the specification, a “user” may be, but is not limited to, a medical expert including a doctor, a nurse, a medical laboratory technologist, a medial image expert, a radiologist, and a technician who repairs a medical apparatus.

The system is described with reference to the accompanying drawings and may be embodied in many different forms and should not be construed as being limited to the embodiments set forth herein; rather, these embodiments are provided so that this disclosure will be thorough and complete, and will fully convey the concept of the invention to those of ordinary skill in the art. In the following description, well-known functions or constructions are not described in detail since they would obscure the invention with unnecessary detail. Throughout the specification, like reference numerals in the drawings denote like elements.

As used herein, the term “and/or” includes any and all combinations of one or more of the associated listed items. Expressions such as “at least one of,” when preceding a list of elements, modify the entire list of elements and do not modify the individual elements of the list.

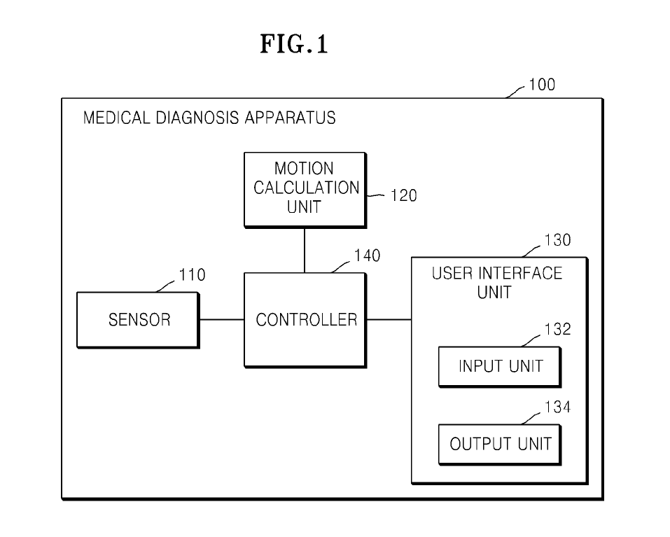

FIG. 1 shows medical imaging apparatus 100 that may include a sensor 110, a motion calculation unit 120, a user interface unit 130, and a controller 140. The medical imaging apparatus 100 performs a medical diagnosis process on an examinee and may generate and output a medical image by scanning the examinee. The medical imaging apparatus 100 may include at least one of an MRI apparatus, a CT apparatus, and an X-ray apparatus. The medical imaging apparatus 100 may detect a motion of the examinee while diagnosing the examinee by using different methods such as a sensor, a medical image and an image captured by a camera, while diagnosing the examinee. The medical imaging apparatus 100 may provide at least one of a user and the examinee with various types of information according to the motion of the examinee. The sensor 110 detects the motion of the examinee and measures a level of the detected motion of the examinee on a diagnosis table or a cradle for medical diagnosis. The “motion” may mean a change in a physical characteristic value measured with respect to the examinee using different types of sensors and may determine that the “motion” is detected according to the change in the measured physical characteristic value.

The “motion” may be expressed as an “amount” indicating the change in the physical characteristic value. That is, the sensor 110 may detect the “motion” and measure the “level of motion”. For example, the sensor 110 may measure a tilt change of the examinee by using a tilt sensor, to detect the motion. The sensor 110 may measure a pressure change in the diagnosis table of the examinee by using a pressure sensor or measure a frequency size change in an RF signal by using an RF sensor, to detect the motion. The sensor 110 may detect the motion of the examinee by using a plurality of different types of sensors. For example, the sensor 110 may include at least one of an optical sensor such as an infrared sensor, the tilt sensor or the pressure sensor for detecting a change in a position or a pressure of the examinee as described above, and the RF sensor for measuring a change in a frequency or size of a received RF signal. The sensor 110 may further include different units such as an acceleration sensor, a gyro sensor, a magnetic field sensor, for example, to detect the motion of the examinee.

The sensor 110 may detect the motion of the examinee based on the medical image as well as a physical characteristic value and may obtain a difference between image characteristic values by comparing a plurality of medical images obtained via a protocol which is a group of sequential signals or pulses for diagnosing the examinee, and may detect the motion according to a calculated difference value. In response to examinee movement during a medical imaging operation, a motion artifact may occur in a medical image. The sensor 110 may compare the most recently obtained medical image from among generated medical images with other images and may compare a previously stored reference medical image with a currently obtained medical image with respect to a protocol currently being used. Alternatively sensor 110 may compare the previously stored reference medical image with a previously obtained and stored medical image with respect to the protocol currently being used. The reference medical image may be an average image of the plurality of medical images obtained via the protocol or may be an image selected by a user input, from the plurality of medical images obtained via the protocol.

The sensor 110 may detect a blurring phenomenon by which a boundary of a subject in the currently obtained medical image is blurred or may detect a noise that occurs in the currently obtained medical image. Also, the sensor 110 may compare object parts of the examinee in the currently obtained medical image with previously captured medical images, or may compare changes in a vector direction of an object part. The sensor 110 may calculate a difference value by comparing the image characteristic value including brightness, chroma, resolution, a position of a boundary line, comparable objects, for example of the currently obtained medical image with image characteristic values of previously captured medical images, and may detect the motion of the examinee from a motion artifact detected according to the difference value.

The sensor 110 advantageously detects the motion of the examinee from an image obtained by directly observing the examinee by using an image capturing unit. The sensor 110 may include different types of image capturing devices such as an infrared-ray camera, a high-speed camera, a wide viewing angle camera, for example, to provide an image of the examinee. The sensor 110 may detect the motion of the examinee based on the user input received by an input unit 132 of the user interface unit 130 and may automatically detect the motion of the examinee or may manually detect the motion of the examinee when the user input is received. The motion calculation unit 120 may compare the level of the detected motion with a predetermined threshold value relating to a physical characteristic value. For example, when the sensor 110 detects the motion from the pressure change in the diagnosis table of the examinee, the motion calculation unit 120 may compare a physical characteristic value of the pressure change with a previously stored threshold value relating to the pressure.

A previously determined threshold value is compared with a corresponding respective physical characteristic value. The threshold value may also be previously determined and dynamical adapted according to a type of imaging protocol that is used with respect to an object. That is, for the same physical characteristic value (for example, a pressure), different threshold values may be determined with respect to different imaging protocol. A first imaging protocol may require a relatively accurate measurement result compared to a different second imaging protocol, for example. For example, the motion calculation unit 120 may employ different threshold values for a scout scan and a brain scan. For another example, the threshold value may be determined according to a position in K-space from which medical image data is obtained. That is, the motion calculation unit 120 may set a lower threshold value (i.e. a less accurate comparison) for data in a center portion in the K-space and a higher threshold value (i.e. a less accurate comparison) for data in a boundary portion in the K-space.

The user interface unit 130 provides the user with different types of information regarding a diagnosis of the examinee, and receives an input to control the medical imaging apparatus 100 from the user. The user interface unit 130 includes the input unit 132 and an output unit 134. The user interface unit 130 may provide the user with different types of information by outputting a generated medical image on a screen of the medical imaging apparatus 100 or by outputting guide information in a graphical form or a text form to the user. In addition, the user interface unit 130 may output information regarding an ongoing imaging protocol to the screen and may provide the user with information regarding a part that is diagnosed.

The input unit 132 comprises a unit used by the user to input data for controlling the medical imaging apparatus 100. For example, the input unit 132 may include, but is not limited to, a key board, a mouse, a dome switch, a touch pad (a touch capacitive type touch pad, a pressure resistive type touch pad, an infrared beam sensing type touch pad, a surface acoustic wave type touch pad, an integral strain gauge type touch pad, a piezo effect type touch pad, for example), a jog wheel, a jog switch, for example. In particular, when the touch pad and a display panel form a layer structure, this may be a touch screen. The input unit 132 may detect not only a real touch but also detect a proximity touch. The input unit 132 may detect a touch input (e.g., a touch & hold input, a tap input, a double-tap input, a flick input, for example) with respect to the output guide information. The input unit 132 may detect a drag input from a point in which the touch input is detected. The input unit 132 may detect multiple touch inputs (e.g., a pinch) with respect to at least two points of the guide information. The output unit 134 outputs different types of information generated and processed by the medical imaging apparatus 100 and provides the information to the user. The output unit 134 according to an embodiment may include a video output unit (not shown) for outputting a video signal and an audio output unit (not shown) for outputting an audio signal.

The video output unit displays and outputs different types of information processed by the medical imaging apparatus 100. For example, the video output unit may output different types of information related to a diagnosis of the examinee, such as a generated medical image, information regarding an ongoing imaging protocol and guide information used to guide the examinee, for example. However, the information displayed on a screen by the video output unit is exemplary. The video output unit may output other types of information to the screen. The “guide information” is information used to guide the examinee relating to the detected motion. In more detail, the guide information may be information regarding a command or an instruction determined according to the level of motion of the examinee. For example, the guide information may be information used to instruct the examinee not to move, and may include a message by using at least one of graphical data, text data, and audio data. The output unit 134 may output the guide information expressed as text data, graphical data, and audio data by using the video output unit and the audio output unit.

The video output unit may output different types of information such as the level of the detected motion of the examinee, information regarding the threshold value and information indicating that recapture is necessary, as the guide information. The video output unit may output information regarding a motion direction, a position, and an angle as the guide information used to move the diagnosed part of the examinee. The video output unit may further output an exemplary image regarding the diagnosed part of the examinee and an image obtained by capturing the diagnosed part of the examinee. The output unit 134 provides the guide information in response to a comparison result of the motion calculation unit 120. If the level of the detected motion of the examinee is higher than the threshold value, the output unit 134 may provide the guide information that guides the examinee to stop moving since the motion of the examinee affects a diagnosis.

If the level of the detected motion of the examinee is lower than the threshold value, the output unit 134 may provide the guide information indicating that a minor motion that does not affect the diagnosis is detected. The output unit 134 may further provide guide information that more strongly advises the examinee not to move. The output unit 134 may not provide guide information regarding the motion, since it is difficult for an examinee to remain stationary for the duration of an MRI examination, for example, due to respiration and discomfort. Further, the output unit 134 may not provide guide information concerning motion below the threshold value. The output unit 134 may output information regarding the threshold value as well as the guide information for guiding the examinee.

The output unit 134 may output the guide information both to the user positioned in a console room and to the examinee positioned in a shield room. When the video output unit is formed as a touch screen, the video output unit may be used as not only an output means but also may be used as an input means. That is, the video output unit may be an input means for receiving a touch input via a stylus pen or a part of the human body and may concurrently be an output means for outputting information. The video output unit may include at least one of a liquid crystal display (LCD), a thin-film transistor LCD (TFT LCD), an organic light-emitting diode (OLED) display, a flexible display, and a three-dimensional (3D) display. The medical imaging apparatus 100 may include two or more video output units. The video output unit may include an in-bore display that outputs an image in a bore of the medical imaging apparatus 100.

The audio output unit outputs information in the form of audio data to be provided to the user. For example, the audio output unit may output different types of information including a notice message indicating detection of the motion, a message indicating a process or an end of the imaging protocol and the guide information for guiding the examinee, for example. The audio output unit may also output audio data by using an alarm sound or pre-stored audio data.

The controller 140 controls the operations of the medical imaging apparatus 100. For example, the controller 140 may control the motion calculation unit 120 to compare the information regarding the motion of the examinee detected by the sensor 110 with the threshold value. The controller 140 may also control the output unit 134 to provide the guide information according to the comparison result of the motion calculation unit 120.

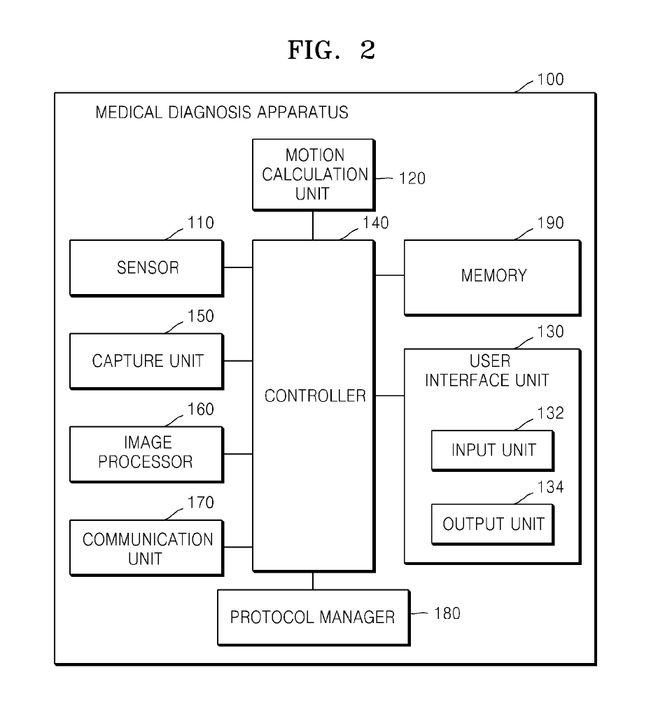

FIG. 2 shows medical imaging apparatus 100 including the capturing unit 150, an image processor 160, a communication unit 170, a protocol manger 180, and a memory 190. Redundant descriptions between FIGS. 1 and 2 are omitted. The capturing unit 150 captures an image of a specific object related to the information providing method performed by the medical imaging apparatus 100. For example, the capturing unit 150 may capture an image of a part of an examinee that is to be diagnosed and generates an image of the diagnosed part. The capturing unit 150 may include at least one of, an infrared camera, a high-speed camera, and a wide viewing angle camera. The capturing unit 150 may be disposed in a shield room in which the medical imaging apparatus 100 is positioned or in a bore of the medical imaging apparatus 100, or may be directly attached to an RF coil and the examinee. The sensor 110 may detect a motion of the examinee by analyzing an image captured by the capturing unit 150. The output unit 134 may provide the image captured by the capturing unit 150 as guide information. The image processor 160 generates a medical image of the object. The image processor 160 generates the medical image by processing medical data obtained by scanning the object. For example, the image processor 160 generates the medical image by processing image data of different types of modalities including X-ray image, a CT image, an MRI image, for example.

The communication unit 170 is connected to a network by wire or wirelessly, to communicate with an external device or a server. The communication unit 170 may communicate data with a hospital server, an external server and an external device, for example, via a picture archiving and communication system (PACS). The communication unit 170 communicates data in a format compatible with the Digital Imaging and Communications in Medicine (DICOM) standard. The communication unit 170 may transmit and receive the medical image over the network and may receive a data request signal or a device control command. The communication unit 170 may also transmit and receive a medical image captured by an apparatus other than the medical imaging apparatus 100.

The communication unit 170 may include one or more elements for enabling communication with an external device. For example, the communication unit 170 may include a short-distance communication module, a wired communication module, and a mobile communication module. The short-distance communication module means a module for short-distance communication within a predetermined distance. Examples of the short-distance communication module may include, but are not limited to, wireless LAN, Wi-Fi, Bluetooth, Zigbee, Wi-Fi Direct (WFD), ultra wideband (UWB), infrared Data Association (IrDA), a Bluetooth Low Energy (BLE), and Near Field Communication (NFC). The wired communication module comprises a module communicating using an electrical signal or an optical signal. Wired communication may involve a cable pair, a coaxial cable, an optical fiber cable or an Ethernet cable, for example. The wireless communication module exchanges a wireless signal with at least one of a base station, an external terminal, and a server in a mobile communication network. The wireless signal may include different types of data according to a voice call signal, a video call signal, or an exchange of text/multimedia message.

The protocol manager 180 manages an imaging protocol used to capture an image of the examinee. The protocol manager 180 may start a imaging protocol to capture an image of a predetermined part of the examinee, may delay resuming the imaging protocol, may suspend an ongoing imaging protocol, or may resume a suspended imaging protocol. In response to an external input signal identifying a target part to be imaged, the imaging protocol manager 180 determines an imaging protocol to image the particular anatomical. The imaging protocol manager 180 may also obtain information regarding a pulse sequence included in the imaging protocol for imaging the target part. The imaging protocol manager 180 may control a process of the pulse sequence included in the imaging protocol to start, suspend, or resume the pulse sequence.

The imaging protocol manager 180 may also control the process of the imaging protocol according to a predetermined standard. For example, the imaging protocol manager 180 may suspend the imaging protocol when the motion of the examinee is detected, or may suspend the imaging protocol when a level of the detected motion is equal to or greater than a predetermined threshold value. Alternatively, when the level of the detected motion changes to be equal to or less than the predetermined threshold value, or when an external input signal is received, the imaging protocol manager 180 may resume the suspended imaging protocol.

The imaging protocol manager 180 may sequentially proceed with the one or more pulse sequences included in the imaging protocol, or may select some pulse sequences and may proceed with the selected pulse sequences. The imaging protocol manager 180 may extract at least one pulse sequence from among the one or more pulse sequences included in the imaging protocol in response to a standard or an external input signal, and image using the extracted pulse sequence. Alternatively, the imaging protocol manager 180 may determine an order and a list of pulse sequences for a capturing operation, and may sequentially proceed with image acquisition using the determined pulse sequences. The memory 190 stores different types of data generated and processed by the medical imaging apparatus 100. For example, the memory 190 may store information regarding the motion of the examinee detected by the sensor 110. The memory 190 may store information regarding threshold values used by the motion calculation unit 120 by matching the threshold values and physical characteristic values. Furthermore, the memory 190 may store the medical image generated by the medical imaging apparatus 100.

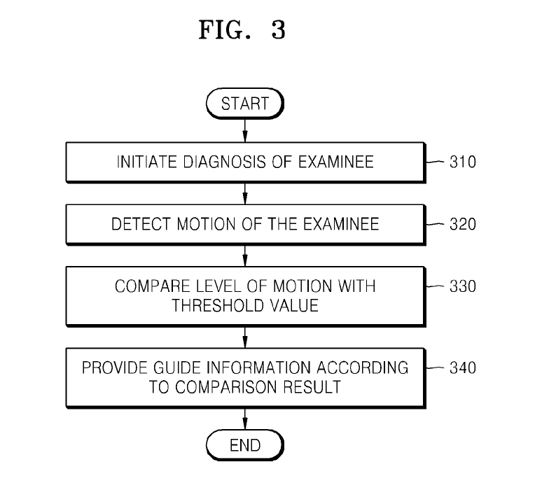

FIG. 3 shows a flowchart of an information providing method using the structure of the medical imaging apparatus 100. Operations are time serially processed by the sensor 110, the motion calculation unit 120, the user interface unit 130, the controller 140, the capturing unit 150, the image processor 160, the communication unit 170, the imaging protocol manager 180, and the memory 190. Thus, hereinafter, although omitted, the descriptions of elements with respect to FIGS. 1 and 2 apply to the flowcharts of FIGS. 3 through 7. In operation 310, the medical imaging apparatus 100 initiates imaging of an examinee. If the examinee is placed on a diagnosis table of the medical imaging apparatus 100 or a cradle, the medical imaging apparatus 100 initiates a selected imaging protocol to image the examinee. The medical imaging apparatus 100 may automatically initiate the imaging if the examinee moves to a pose and position suitable for the imaging, and the imaging may be manually resumed by a user input. The medical imaging apparatus 100 may image an object by using different devices such as an RF coil or an X-ray director in operation 310.

In operation 320, the medical imaging apparatus 100 detects motion of the examinee using different types of sensors. For example, the medical imaging apparatus 100 may measure a change in a physical characteristic value by using a tilt sensor, a pressure sensor, an optical sensor, an acceleration sensor, an RF sensor, for example, that are disposed around the imaging table and may detect the motion of the examinee. The medical imaging apparatus 100 may detect the motion of the examinee by comparing a plurality of acquired medical images. In operation 330, the medical imaging apparatus 100 compares a level of the detected motion with a threshold value. The medical imaging apparatus 100 may compare a level of motion measured in operation 330 with a previously determined threshold value. The medical imaging apparatus 100 may previously match and store threshold values with respect to different types of sensors, and, if the motion is detected, may compare the threshold values with the level of motion.

The threshold value may be previously determined in response to a type of a sensor that detects the motion and in response to a type of the selected imaging protocol for imaging the object. The threshold value may also be determined according to a position in K-space. In operation 340, the medical imaging apparatus 100 provides guide information for guiding the examinee. The medical imaging apparatus 100 may output the guide information in response to a result of comparing the motion and the threshold value in operation 330. The medical imaging apparatus 100 according to an embodiment may provide at least one of a user and the examinee with the guide information by using at least one of graphical data, text data, and audio data.

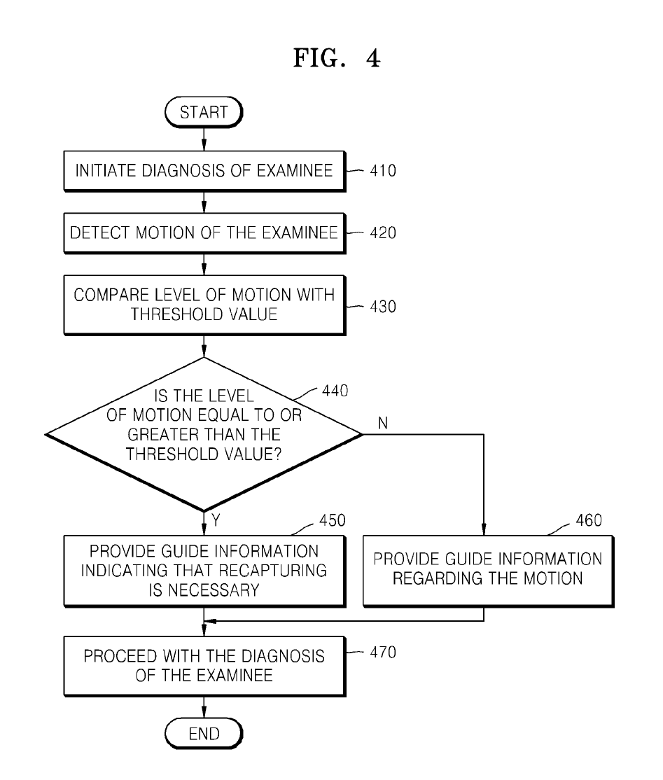

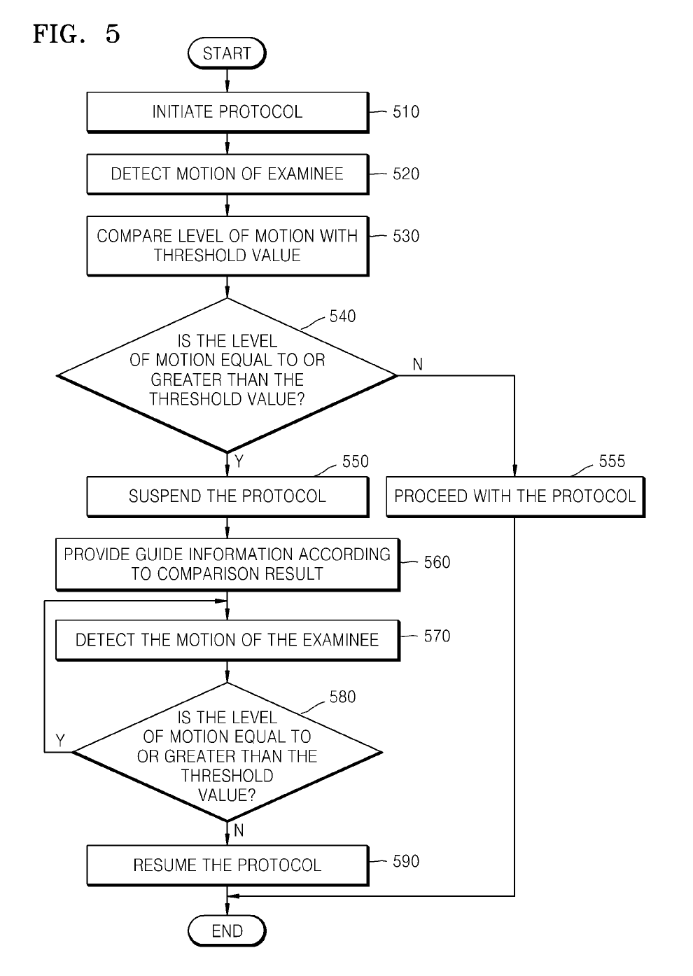

Embodiments regarding operations 330 and 340 will be described in detail with reference to FIGS. 4 and 5. FIG. 4 shows a flowchart of an information providing method. Redundant descriptions between FIGS. 3 and 4 are omitted. In operation 430, the medical imaging apparatus 100 compares a level of a detected motion of an examinee with a threshold value. If it is determined in operation 440 that the level of motion is equal to or greater than the threshold value based on the comparison result, imaging apparatus 100 provides guide information indicating that recapturing is necessary in step 450. The medical imaging apparatus 100 determines that reliability of an imaging result of the examinee may be impaired if the level of motion is equal to or greater than the threshold value. The medical imaging apparatus 100 may provide a user with the guide information to prompt the user to suspend an imaging protocol and control the motion of the examinee. The medical imaging apparatus 100 may display and output the guide information to the user by using graphical data and text data and output the guide information by using audio data.

Alternatively, the medical imaging apparatus 100 may provide the guide information indicating that image recapture is performed because the level of motion of the examinee is excessive to prom the examinee to remain stationary. In operation 460, the medical imaging apparatus 100 indicates that the motion of the examinee is disregarded since the motion is relatively small and equal to or smaller than the threshold value. In operation 460, the medical imaging apparatus 100 may recognize that although the motion is detected, an additional measure, such as suspending of the imaging protocol or recapturing is not required. The medical imaging apparatus 100 may provide the guide information just indicating that the motion is merely detected. For example, if the examinee moves to an extent that exceeds the threshold, the medical imaging apparatus 100 may output the guide information warning that the imaging protocol is suspended, and may output the guide information regarding a result of comparing the detected motion with a threshold value. In operation 470, the medical imaging apparatus 100 may proceed with a imaging on the examinee. That is, the medical imaging apparatus 100 may resume the suspended imaging protocol or may continue to proceed with an ongoing imaging protocol.

FIG. 5 shows a flowchart of an information providing method. In operation 510, the medical imaging apparatus 100 initiates an imaging protocol for imaging of an object. In operation 520, the medical imaging apparatus 100 detects motion of an examinee during execution of the imaging protocol. In operation 530, the medical imaging apparatus 100 compares a level of the detected motion with a threshold value and in operation 540, apparatus 100 determines whether the level of motion is equal to or greater than the threshold value. The apparatus 100 suspends the ongoing imaging protocol if the level of motion is equal to or greater than the threshold value. If the level of motion is equal to or greater than the threshold value, the medical imaging apparatus 100 may expect that the motion of the examinee may interfere with the imaging and so may suspend the imaging protocol.

In operation 555, the medical imaging apparatus 100 continues an imaging protocol in response to the detected motion of the examinee being less than the threshold value and disregards such small motion since the motion negligibly affects the imaging. Alternatively, the medical imaging apparatus 100 may continue with the imaging protocol although the motion of the examinee affects the imaging or a medical image, and corrects image motion artifacts by applying an algorithm in image post-processing. In operation 560, the medical imaging apparatus 100 provides guide information in response to the comparison in the manner previously described in connection with operation 450 of FIG. 4. In operation 570, the medical imaging apparatus 100 detects the motion of the examinee and determines whether the motion of the examinee detected in operation 520 has been reduced to an acceptable level. In operation 580, apparatus 100 determines whether a level of motion of the examinee detected in operation 570 is equal to or greater than the threshold value and apparatus 100 keeps the imaging protocol suspended if the motion of the examinee continues to be equal to or greater than the threshold value and suspends the imaging protocol until the motion of the examinee is smaller than the threshold value. In operation 590, the medical imaging apparatus 100 resumes the suspended imaging protocol if the level of motion of the examinee detected in operation 570 is equal to or smaller than the threshold value. Apparatus 100 waits to resume the imaging protocol until the motion of the examinee does not impair imaging and minimizes effect of motion artifacts on the medical image And may also resume the suspended imaging protocol in response to a user input.

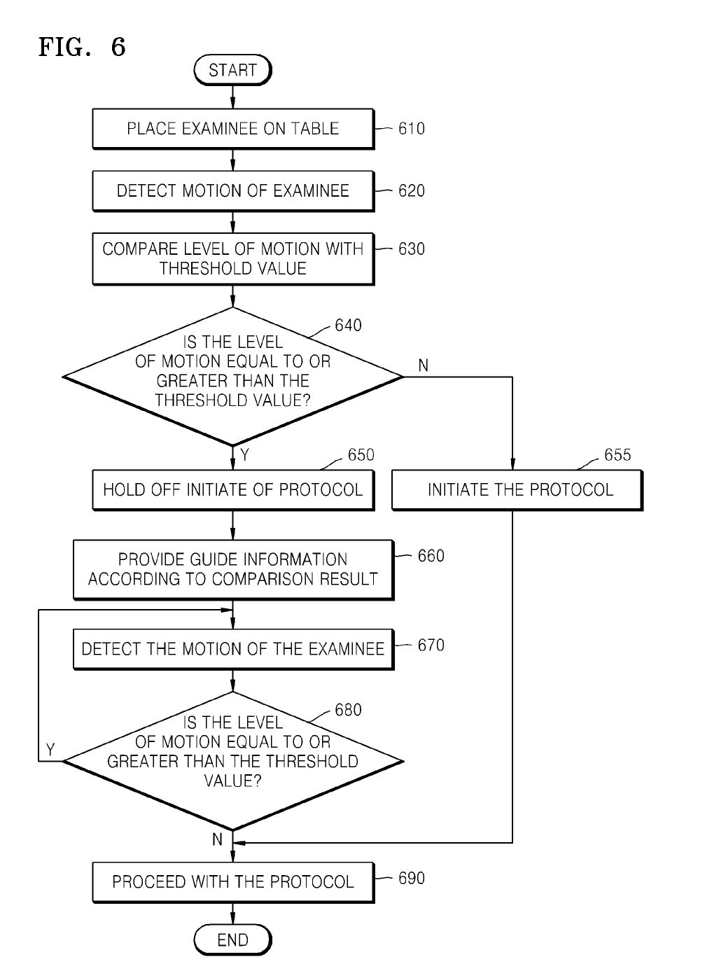

FIG. 6 shows a flowchart of an information providing method where in operation 610, an examinee is placed on a imaging table for medical imaging. In operation 620, the medical imaging apparatus 100 detects motion of the examinee before imaging (for example, an imaging protocol) on the examinee is resumed. In operation 630, the medical imaging apparatus 100 compares a level of the detected motion with a threshold value, and, in operation 640, determines whether the level of the detected motion is equal to or greater than the threshold value. If the level of motion is equal to or greater than the threshold value apparatus 100 in operation 650, delays an initiation of an imaging protocol as the detected motion of the examinee may seriously affect image quality and may wait to start the imaging protocol. If the level of motion is smaller than the threshold value, apparatus 100 initiates execution of the imaging protocol and may disregard the motion of the examinee that is detected to be equal to or smaller than the threshold value.

In operation 660, the medical imaging apparatus 100 provides guide information in response to a result of comparing the motion of the examinee with the threshold value and may provide the guide information for guiding the examinee as described in operation 450 of FIG. 4. In operation 670, the medical imaging apparatus 100 continues to detect the motion of the examinee. In operation 680, the medical imaging apparatus 100 compares the level of the detected motion of the examinee with the threshold value and if the level of motion is equal to or greater than the threshold value, continues to detect the motion and suspend imaging. If the level of motion is equal to or smaller than the threshold value, the level of motion of the examinee may be disregarded and in operation 690 imaging apparatus 100 may resume the imaging protocol held off in operation 650. Imaging apparatus 100 may delay the resumption of the imaging despite a user command to resume the imaging if the motion of the examinee is equal to or greater than the threshold value. Accordingly, the medical imaging apparatus 100 may automatically start the imaging if the motion of the examinee is reduced below the threshold.

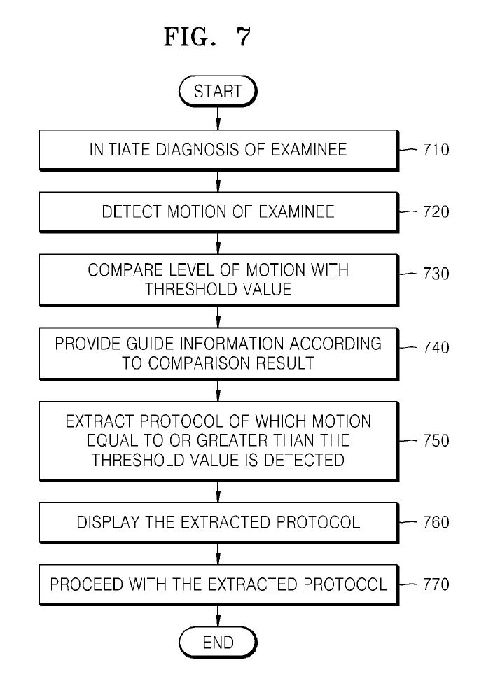

FIG. 7 shows a flowchart of an information providing method. Redundant descriptions between FIGS. 4 and 7 are omitted. In operation 740, the medical imaging apparatus 100 provides guide information in response to comparing a motion of an examinee with a threshold value and indicating that recapture of an image is necessary since a level of motion of the examinee is serious or an imaging protocol is suspended. The medical imaging apparatus 100 may also provide the guide information indicating that although the motion is detected, the detected motion may be disregarded since the detected motion slightly affects the imaging or the detected motion may be corrected through image post-processing. In operation 750, the medical imaging apparatus 100 selects a motion-resistant imaging protocol from a plurality of imaging protocols, if detected motion is equal to or greater than the threshold value. In operation 760, the medical imaging apparatus 100 displays data identifying the imaging protocol used in operation 750. Apparatus 100 may display a marker identifying an imaging protocol of which motion is detected from the plurality of imaging protocols or may display a list of extracted imaging protocols. In operation 770, the medical imaging apparatus 100 automatically proceeds with the imaging protocol extracted in operation 750 after other imaging protocols end or based on a user input.

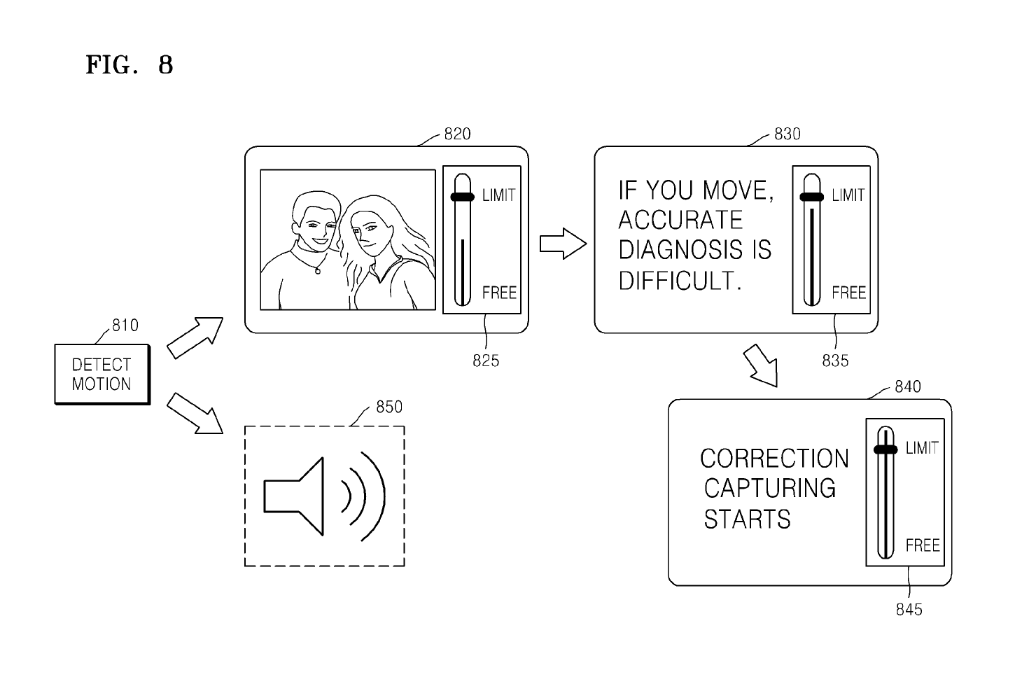

FIG. 8 shows a system for providing guide information in response to a level of a detected motion. The medical imaging apparatus 100 detects a motion of an examinee (810) and provides guide information by using at least one of text data, graphical data, and audio data as shown. The medical imaging apparatus 100 displays an image for attracting examinee attention on output unit 820 during medical imaging. Apparatus 100 displays a gauge 825 comparing a level of the detected motion with a threshold value and may use different types of physical characteristic values as threshold values. For example, the gauge 825 of FIG. 8 may indicate an angle change in a part of the examinee that is diagnosed, a speed change, an acceleration change or an intensity change in an RF signal, for example. Although not shown, the medical imaging apparatus 100 may display a numerical value of the level of the detected motion compared to the threshold value as well as the gauge 825. For example, the medical imaging apparatus 100 may display 60% as a current level of the detected motion compared to the threshold value. Gauge 825 on unit 820 indicates detected motion is equal to or smaller than the threshold value and so does not display a specific instruction or a command to control the motion to the examinee. Gauge 835 on unit 830 indicates detected motion has increased. Apparatus 100 may output guide information comprising text data “if you move, accurate imaging is difficult” to prompt an examinee to reduce motion. Gauge 845 on unit 840 indicates detected motion has further increased. Apparatus 100 may output guide information indicating that the level of motion of the examinee exceeds the threshold value on the output unit 840. The medical imaging apparatus 100 guide information may induce the examinee to control the motion for himself/herself. In an embodiment, apparatus 100 outputs the guide information by using the audio data (850) on the output units 820, 830, and 840 of FIG. 8 by using previously stored audio data.

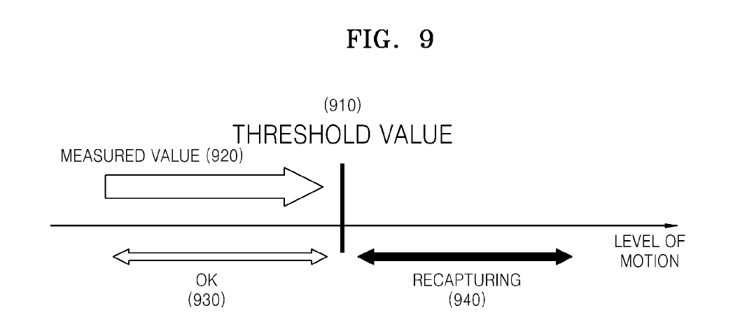

FIG. 9 shows presentation of a threshold value 910 of motion. Apparatus 100 may measure a level of motion of an examinee by using different types of sensors. The medical imaging apparatus 100 may compare the previously stored threshold value 910 with a measured value 920 with respect to a sensor that detects the motion of the examinee. The medical imaging apparatus 100 may recognize that the motion of the examinee is minor if the measured value 920 is equal to or smaller than the threshold value 910 and may continue to proceed with imaging (930) and disregard the motion of the examinee or may remove a motion artifact through post-processing of a medical image. The medical imaging apparatus 100 may recapture an image of the examinee (940) if the measured value 920 is equal to or greater than the threshold value 910 by suspending the imaging and, if the motion is no longer detected or a user input is received, may resume the imaging.

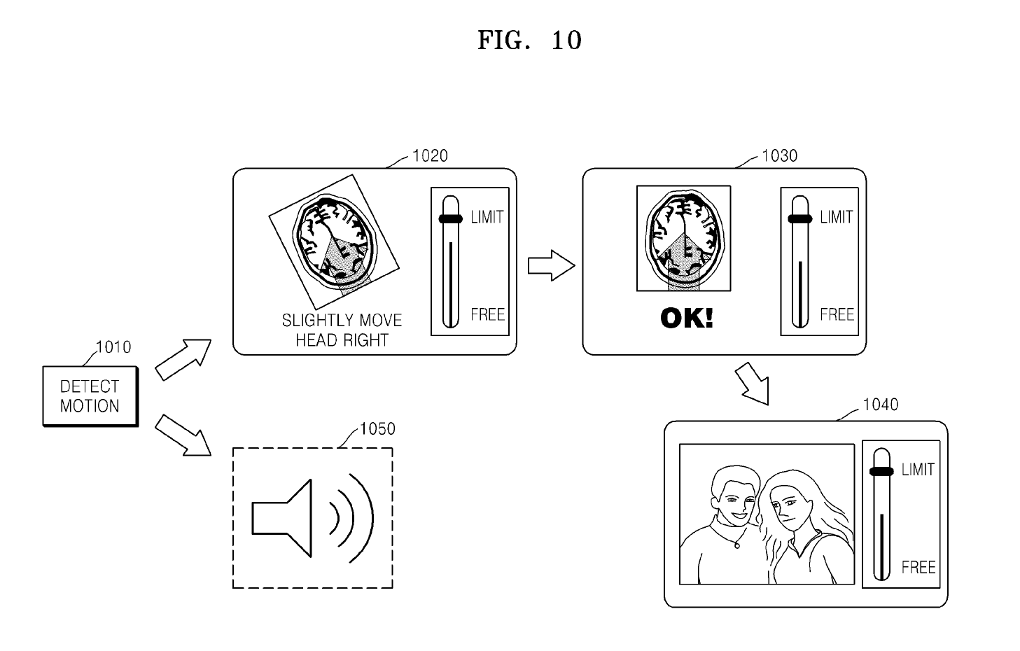

FIG. 10 shows providing guide information by detecting motion of an examinee (1010) and providing guide information. Apparatus 100 displays the guide information used to move an imaging part of the examinee on an output unit 1020. The medical imaging apparatus 100 may accurately detect a position and pose of an examinee brain using a scan of an examinee brain. If an examinee's head moves left from a reference position, the apparatus 100 may provide the guide information to guide the examinee to move his/her head right. The medical imaging apparatus 100 may display a gauge comparing a level of the detected motion with a threshold value on the output unit 1020. The gauge of the present embodiment may be an angle of a imaging part. The medical imaging apparatus 100 may provide different types of information such as a movement direction, a movement distance, a position, etc. in addition to the angle for moving the imaging part of the examinee as the guide information.

Apparatus 100 may detect motion of a head relative to a reference position according to the guide information and display an accurate position and pose of the head on output unit 1030. If the motion of the examinee is reduced or eliminated, the medical imaging apparatus 100 may output an image before further motion is detected on an output unit 1040. Apparatus 100 may output the guide information by using audio data (1050). That is, the medical imaging apparatus 100 may load and output audio data corresponding to the guide information displayed on the output units 1020, 1030, and 1040.

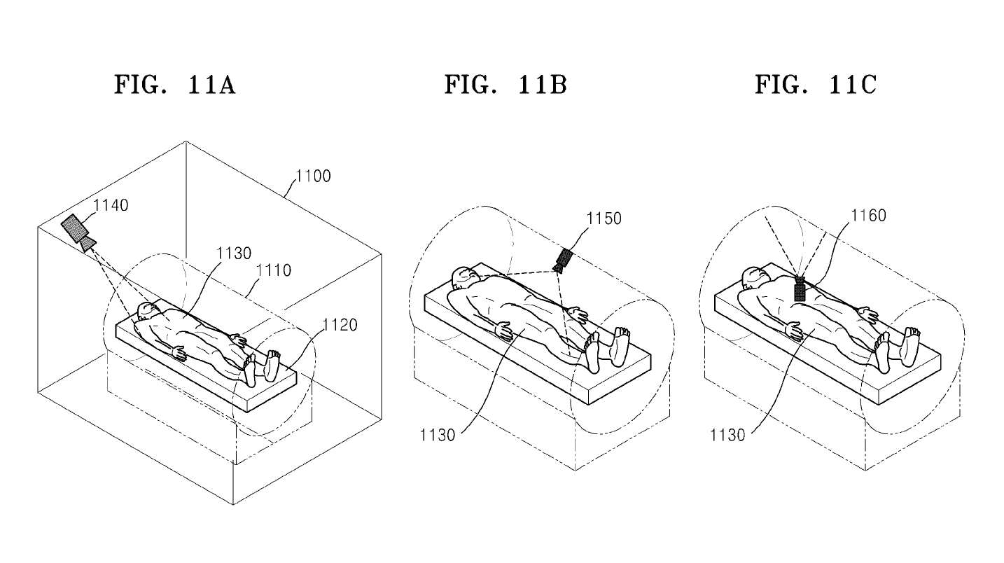

FIGS. 11A through 13 show detection of motion of an examinee 1130 performed by the medical imaging apparatus 100 using cameras 1140, 1150, and 1160, respectively. FIGS. 11A through 11C show the capturing unit 150 that detects the motion of the examinee 1130 at different positions. An MRI apparatus 1110 is disposed in a shield room 1100, and the examinee 1130 is placed on a imaging table 1120. The medical imaging apparatus 100 may include camera 1140 that detects the motion of the examinee 1130 in the shield room 1100 using an image that is captured and generated by the camera 1140. For example, the medical imaging apparatus 100 may detect the motion of the examinee 1130 by measuring a change in a color, chroma, and brightness characteristics of each pixel of a moving image. Alternatively, the motion of the examinee 1130 may provide a user with the image captured by the camera 1140 and may detect the motion of the examinee 1130 based on an input received from the user.

The motion of the examinee 1130 is detected by the camera 1150 disposed in the MRI apparatus 1110. The camera 1150 may capture the image of the examinee 1130 and detect the motion of the examinee 1130 from a captured image as described with reference to FIG. 11A. The cameras 1140 and 1150 of FIGS. 11A and 11B may be replaced with an optical sensor such as an infrared sensor, and may use other different sensors described with reference to FIG. 1. The camera 1160 may be attached to the examinee 1130. That is, the camera 1160 of FIG. 11C does not capture an image of the examinee 1130 but captures an image of an inside of a bore included in the MRI apparatus 1110. The medical imaging apparatus 100 may detect if the examinee moves by analyzing an image of the inside of the MRI apparatus 1110 captured by the camera 1160 by using a landmark identifying an image of the inside of the bore. In addition, the camera 1160 may be attached to an RF coil to capture an image of the inside of the MRI apparatus 1110.

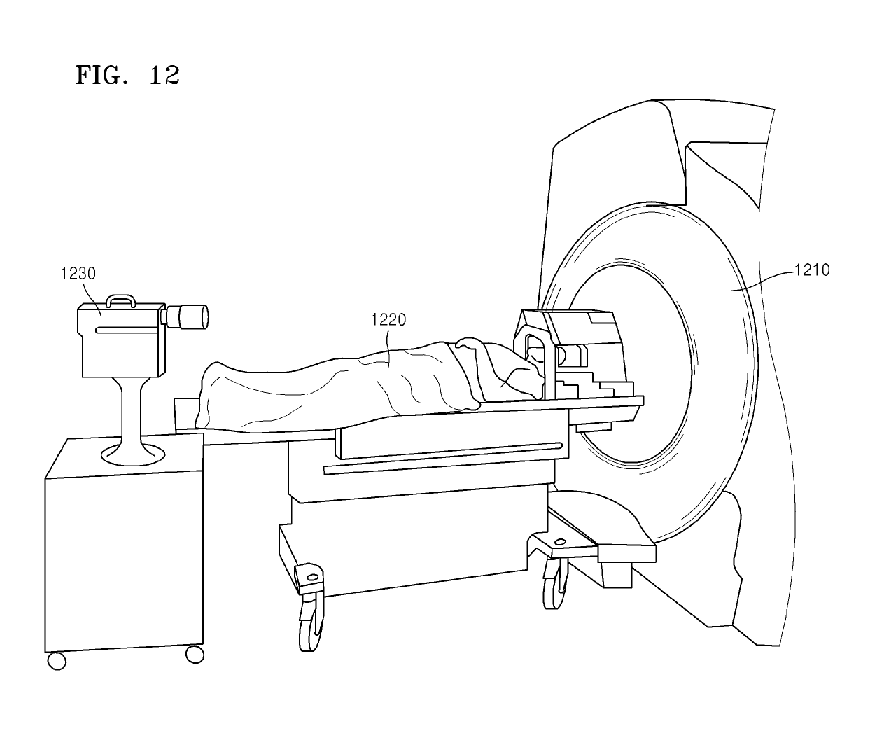

FIG. 12 shows detection of a motion of an examinee 1220 by using a camera 1230 in a CT apparatus 1210 that images the examinee 1220 that is placed on a imaging table and moved into a bore. Alternatively, the medical imaging apparatus 100 may capture a movement path of the imaging table on which the examinee 1220 is placed. The medical imaging apparatus 100 may detect the motion of the examinee 1220 from an image of the examinee 1220 or the movement path of the imaging table and may detect if the motion of the examinee exists by analyzing the image captured by the camera 1230 as described with reference to FIG. 11. The medical imaging apparatus 100 may also detect a level of motion of the examinee 1220.

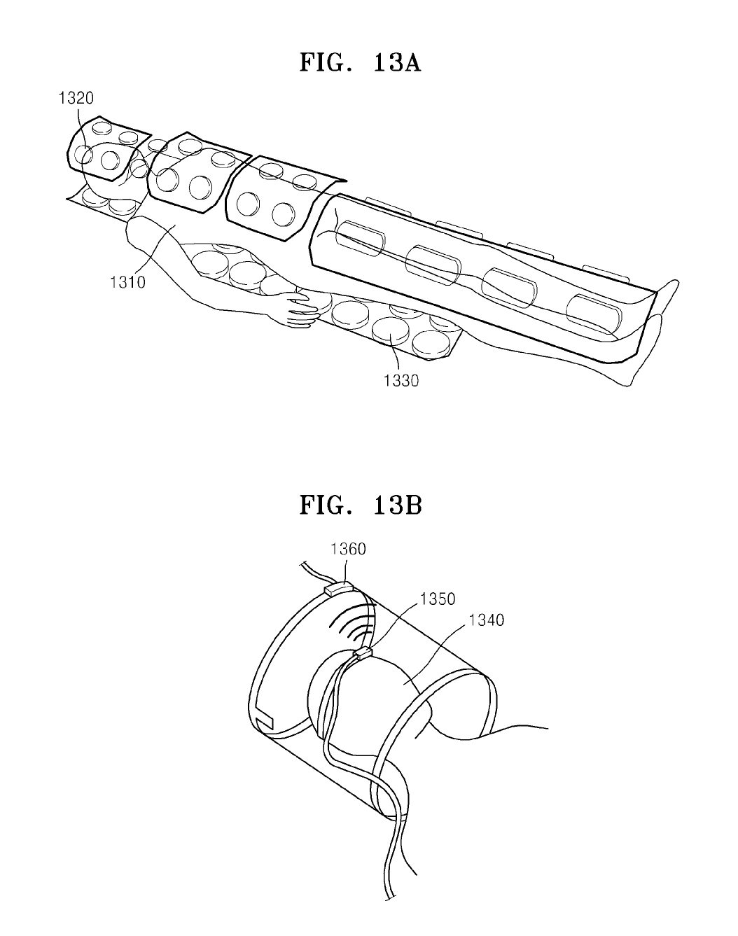

FIGS. 13A and 13B show detection of motion of examinees 1310 and 1340 by using different types of sensors 1320, 1330, and 1360, respectively. FIG. 13A shows the piezoelectric sensors 1320 and 1330. FIG. 13B shows an RF sensor 1360. The medical imaging apparatus 100 detects the motion of the examinee 1310 by using the piezoelectric sensors 1320 and 1330 attached to the examinee 1310. The piezoelectric sensors 1320 and 1330 may detect the motion of the examinee 1310 and convert the motion into an electrical signal. Accordingly, the medical imaging apparatus 100 may measure a change in an intensity of a signal received through the piezoelectric sensors 1320 and 1330 or a frequency thereof and may detect a motion of a predetermined part of the examinee 1310.

Referring to FIG. 13B, the medical imaging apparatus 100 detects the motion of the examinee 1340 by using the RF sensor 1360. That is, the medical imaging apparatus 100 receives an RF signal transmitted from an identification device 1350 attached to the examinee 1340 through the RF sensor 1360. Thereafter, the medical imaging apparatus 100 may detect the motion of the examinee 1340 from a change in an intensity of a signal received through the RF sensor 1360 or a frequency thereof.

The medical imaging apparatus 100 may detect a motion of an examinee by using a plurality of sensors. That is, the medical imaging apparatus 100 may detect the motion from a change in a physical characteristic value measured by each of the plurality of sensors. Furthermore, the medical imaging apparatus 100 may detect a direction of the motion from one or more sensors of which characteristic values are changed from among the plurality of sensors. The medical imaging apparatus 100 may use different types of sensors in addition to the sensors described with reference to FIGS. 13A and 13B as described above. The medical imaging apparatus 100 may measure a change in a physical characteristic value by using a sensor and may detect the measured change as a motion of an examinee. Furthermore, the medical imaging apparatus 100 may compare a threshold value previously matching the physical characteristic value with the measured change and may determine whether a level of motion of the examinee is equal to or greater than the threshold value.

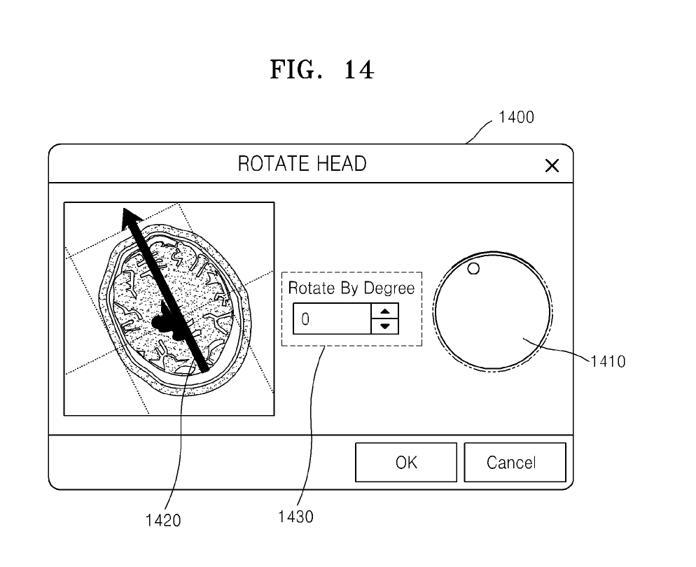

FIG. 14 is a diagram for explaining about providing of an examinee with guide information regarding a motion. The medical imaging apparatus 100 provides the guide information for guiding the examinee, on an output unit 1400. As described above, the medical imaging apparatus 100 may provide at least one of a user and the examinee with the guide information. The medical imaging apparatus 100 may capture a medical image of an examinee's head and may detect that the examinee's head has rotated during the capturing. The medical imaging apparatus 100 detects a motion of the examinee's head and outputs the guide information to guide the examinee to rotate his/her head at an angle suitable for the capturing. The medical imaging apparatus 100 may indicate a line 1420 on an image of the examinee and may display a relationship between a reference angle and a rotated angle of the examinee's head. The medical imaging apparatus 100 may display information regarding a rotational angle of the examinee's head by using graphical data 1410 and text data 1430.

The medical imaging apparatus 100 may identify and display the angle of the examinee's head for capturing from an unsuitable angle by using the graphical data 1410. Accordingly, the medical imaging apparatus 100 may provide a different type of guide information based on an angle section including the angle of the examinee's head. For example, the medical imaging apparatus 100 may provide guide information indicating that capturing starts and is resumed if the angle of the examinee's head is included in a reference angle section. If the angle of the examinee's head is included in a section other than the reference angle section, the medical imaging apparatus 100 may provide guide information indicating that rotation of the angle of the examinee's head is necessary as well as guide information indicating that the capturing is suspended.

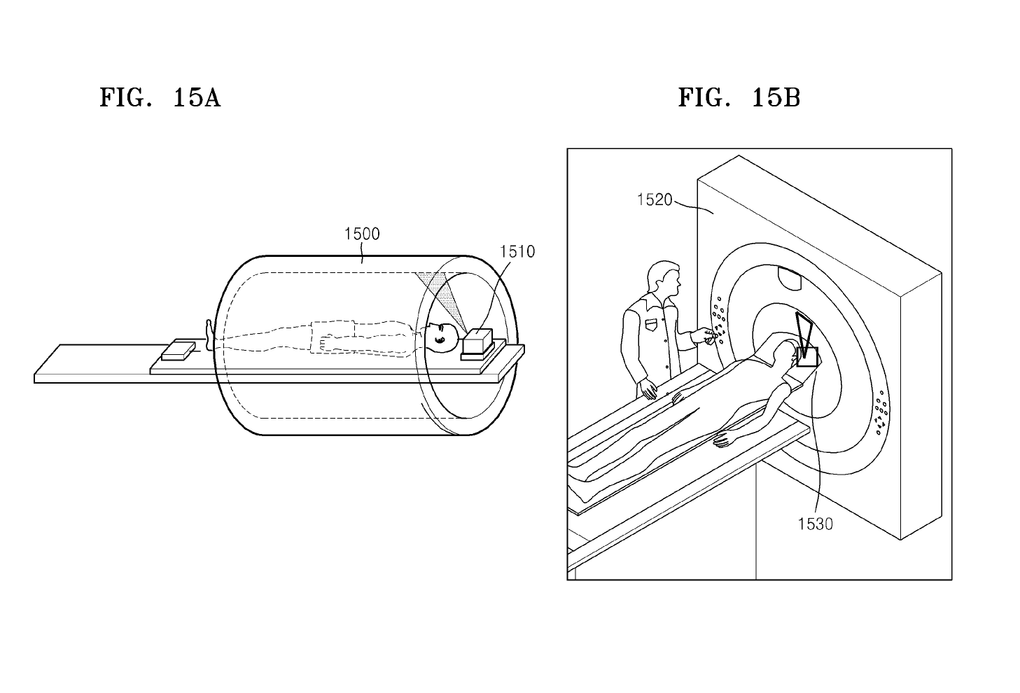

FIGS. 15A and 15B show a system for providing guide information through in-bore displays 1510 and 1530 used in MRI apparatus 1500 and CT apparatus 1520 respectively. Apparatus 100 may use the in-bore display 1510 to display and output the guide information in a gantry of the MRI apparatus 1500. Apparatus 100 may display the guide information on the in-bore display 1530 in a gantry of the CT apparatus 1520. The medical imaging apparatus 100 may project an image-forming beam onto an inner wall of the gantry. The inner wall of the gantry may function as a screen for the projected image-forming beam.

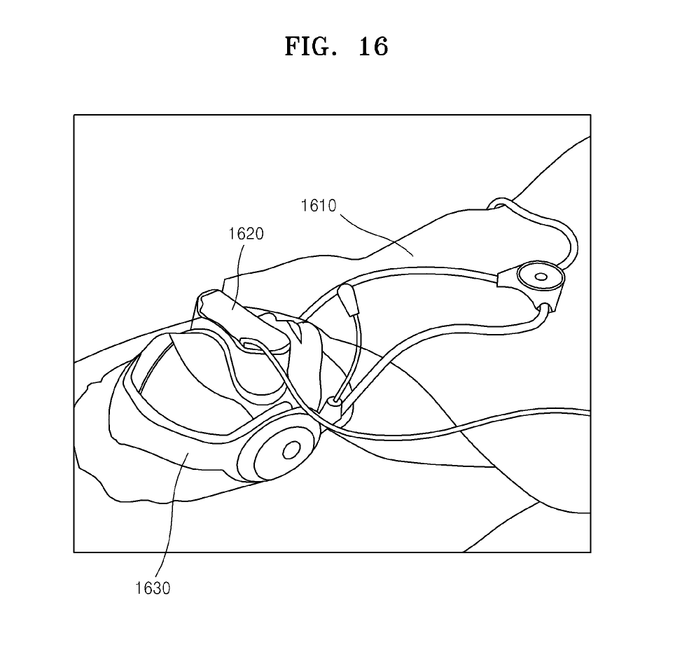

FIG. 16 shows a system for providing an examinee 1610 with guide information. The medical imaging apparatus 100 may use a head-mount display 1620 in providing the examinee 1610 with the guide information. The medical imaging apparatus 100 may output the guide information on the head-mount display 1620 and may provide image data for guiding a motion of the examinee 1610. The medical imaging apparatus 100 may provide the examinee 1610 with audio data by using a headset 1630 or using other different types of output units.

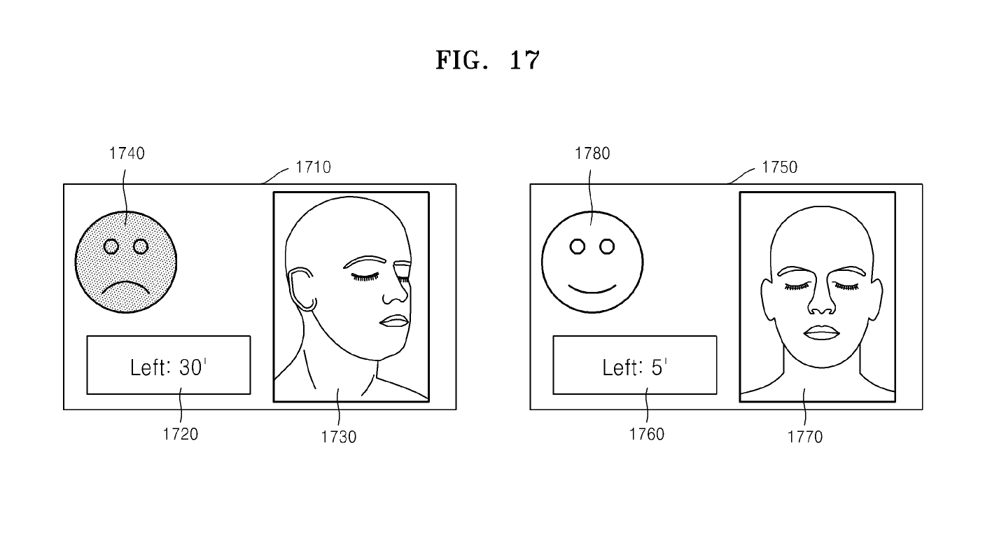

FIG. 17 shows a system providing guide information using an image of an examinee. Apparatus 100 captures an image of the examinee head or other anatomical part by using a camera as described with reference to FIGS. 11 and 12. Apparatus 100 detects a motion of the examinee's head and may detect that the examinee's head rotates to the left by about 30 degrees from a reference position for example. Apparatus 100 may display the guide information indicating that rotation of the examinee's head to a position suitable for imaging is necessary. The medical imaging apparatus 100 may display information 1720 regarding the motion of the examinee, i.e., a rotation angle of the examinee's head and a rotation direction thereof, on an output unit 1710.

Apparatus 100 may display an image 1730 of the examinee's head as the guide information and may display the image 1730 of the examinee's head that is the imaging part of the examinee as well as the information 1720 regarding the motion of the examinee. As shown, since the motion of the examinee is equal to or greater than a threshold value, the medical imaging apparatus 100 may display a symbol 1740 indicating that recapturing is necessary as the guide information. Apparatus 100 may detect that the angle and direction of the examinee's head are adjusted. and rotated to the left by about 5 degrees from the reference position, for example. The medical imaging apparatus 100 may display an image 1770 of the examinee's head concurrently with the displaying of information 1760. If the motion of the examinee is detected to be smaller than the threshold value, the medical imaging apparatus 100 may display a symbol 1780 indicating that it is unnecessary to suspend or delay the imaging as the guide information.

The system advantageously provides the examinee or a user with the guide information regarding the motion. That is, the examinee and the user may easily compare a position of a current imaging part with a position suitable for the imaging. The medical imaging apparatus 100 may display a previously stored exemplary image, other than a directly captured image of the examinee, as the guide information, unlike FIG. 17. That is, the medical imaging apparatus 100 may previously store an exemplary image matching each imaging part and imaging protocol, and, if the motion of the examinee is detected, may load and display the exemplary image corresponding to a level of the detected motion.

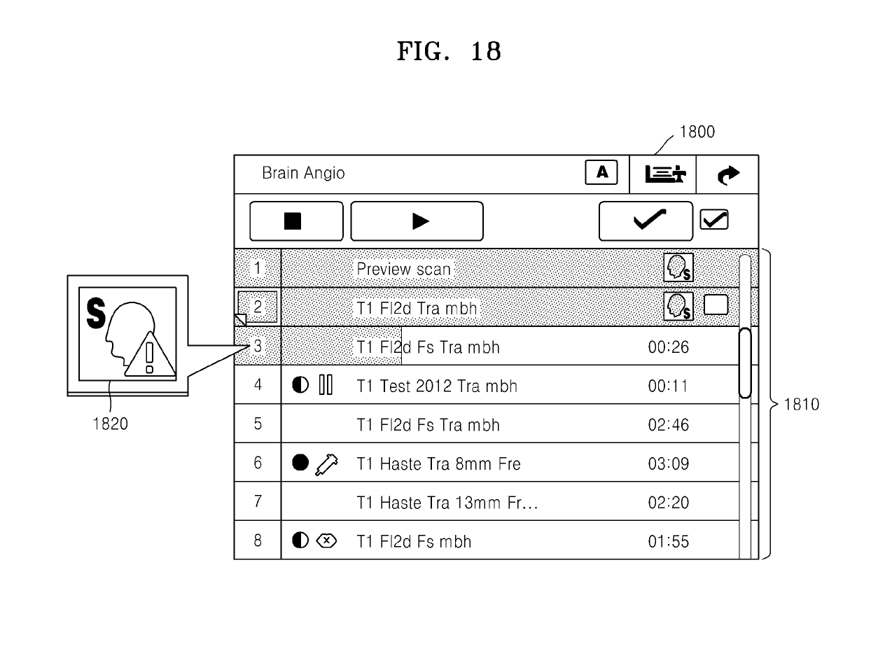

FIG. 18 adaptive selection of an imaging protocol (pulse sequence) in response to detected motion. Apparatus 100 may extract and display on unit 1800 the imaging protocol of which motion is detected from a plurality of imaging protocols 1810. Apparatus 100 may detect that a level of motion of the examinee is equal to or greater than a threshold value while executing a third imaging protocol as indicated by symbol (“!”) 1820. Apparatus 100 may selectively extract the imaging protocol of which motion is detected from the plurality of imaging protocols 1810. In response to completion of the imaging protocols 1810, apparatus 100 prompts a user to employ an imaging protocol to recapture an image during previous capture of which motion was detected. The apparatus 100 may proceed with the imaging protocol of which motion is detected automatically or according to a user input after proceeding completion of imaging protocols 1810.

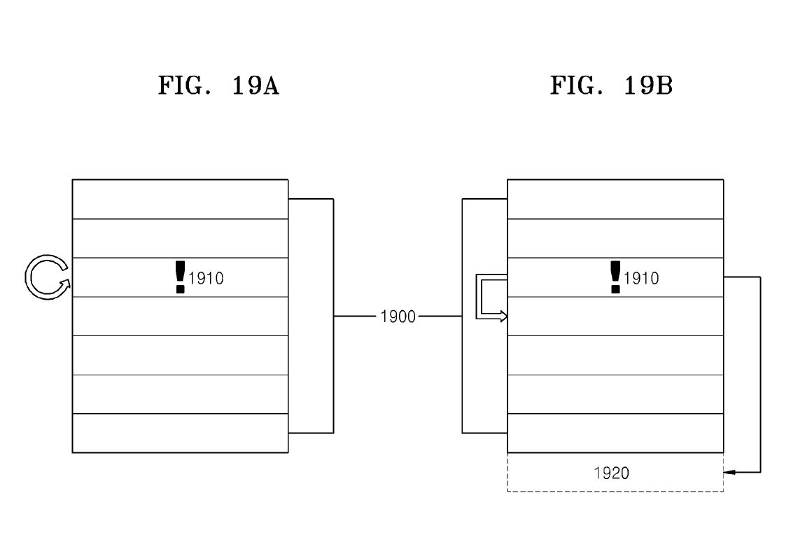

FIGS. 19A and 19B show resuming of suspended imaging protocols where blocks 1900 indicate imaging protocols. Referring to FIG. 19A, the medical imaging apparatus 100 detects a motion of an examinee while executing a third imaging protocol 1910. The medical imaging apparatus 100 may suspend the third imaging protocol 1910, and, if the motion of the examinee is detected to be equal to or smaller than a threshold value, may resume the third imaging protocol 1910. Apparatus 100 detects the motion of the examinee while executing the third imaging protocol 1910. Unlike FIG. 19A, the medical imaging apparatus 100 may skip the third imaging protocol 1910 of which motion is detected and may start a fourth imaging protocol. The medical imaging apparatus 100 may execute the third imaging protocol 1910 of which motion is detected after completion of imaging protocols (1920) automatically or according to a user input.

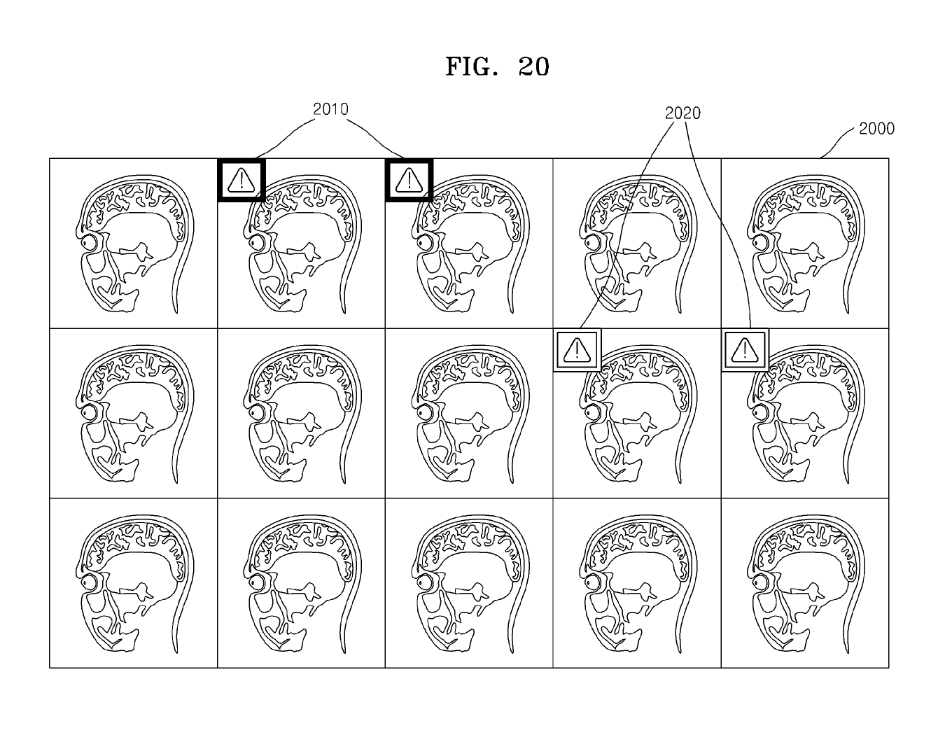

FIG. 20 shows display of medical images 2000 in which motion is detected. A plurality of medical images of FIG. 20 are obtained by capturing sagittal view images of an examinee's head. The medical imaging apparatus 100 may display a marker on the medical image during acquisition of which motion is detected. Apparatus 100 displays a marker 2010 with a black frame on an image captured through an imaging protocol of which motion equal to or greater than a threshold value is detected. The medical imaging apparatus 100 may display a marker 2020 with a white frame on an image captured through a imaging protocol of which motion equal to or smaller than the threshold value is detected. That is, the medical imaging apparatus 100 may use the marker 2020 with the white frame to display that although a motion of an examinee is detected, since the motion does not seriously affect a imaging, the imaging protocol is not suspended. Although not shown, the medical imaging apparatus 100 may use a visually different marker to display an image in which motion is compensated through image post-processing. Apparatus 100 may allow a user to determine a medical image requiring recapture among a plurality of medical images. Apparatus 100 may extract a list of images in which there is motion equal to or greater than the threshold value from the obtained medical images and display the list on a screen, unlike FIG. 20. A user of the medical imaging apparatus 100 may easily identify a medical image in which motion is detected and determine whether to perform image recapture.

The embodiments of the present invention may be written as computer programs and may be implemented in general-use digital computers that execute the programs using a computer-readable recording medium. In addition, a data structure used in the embodiments of the present invention may be written in a computer-readable recording medium through different means. Examples of the computer-readable recording medium include magnetic storage media (e.g., ROM, floppy disks, hard disks, etc.), optical recording media (e.g., CD-ROMs, or DVDs), etc.

A user and an examinee may advantageously identify motion of the examinee detected during imaging using guide information regarding the motion of the examinee, thereby efficiently and actively supporting a medical imaging.

The user may control a medical imaging process by using information regarding the motion of the examinee, and may carry out recapture according to circumstances. The examinee may recognize an effect of the motion of the examinee on capture, may minimize the effect by correcting a pose and a position guided by the guide information, and may correct the pose and the position according to an instruction from the user.

While this system has been particularly shown and described with reference to exemplary embodiments thereof, it will be understood by those of ordinary skill in the art that different changes in form and details may be made therein without departing from the spirit and scope of the invention. The exemplary embodiments should be considered in a descriptive sense only and not for purposes of limitation. Therefore, the scope of the invention is defined not by the detailed description of the invention but by the appended claims, and differences within the scope will be construed as being included in the present invention.

The above-described embodiments can be implemented in hardware, firmware or via the execution of software or computer code that can be stored in a recording medium such as a CD ROM, a Digital Versatile Disc (DVD), a magnetic tape, a RAM, a floppy disk, a hard disk, or a magneto-optical disk or computer code downloaded over a network originally stored on a remote recording medium or a non-transitory machine readable medium and to be stored on a local recording medium, so that the methods described herein can be rendered via such software that is stored on the recording medium using a general purpose computer, or a special processor or in programmable or dedicated hardware, such as an ASIC or FPGA. As would be understood in the art, the computer, the processor, microprocessor controller or the programmable hardware include memory components, e.g., RAM, ROM, Flash, etc. that may store or receive software or computer code that when accessed and executed by the computer, processor or hardware implement the processing methods described herein. In addition, it would be recognized that when a general purpose computer accesses code for implementing the processing shown herein, the execution of the code transforms the general purpose computer into a special purpose computer for executing the processing shown herein. The functions and process steps herein may be performed automatically or wholly or partially in response to user command. An activity (including a step) performed automatically is performed in response to executable instruction or device operation without user direct initiation of the activity. No claim element herein is to be construed under the provisions of 35 U.S.C. 112, sixth paragraph, unless the element is expressly recited using the phrase “means for.”

What is claimed is:

1. A method of providing medical imaging guidance information for use in operating a medical imaging apparatus, the method comprising:
detecting motion of an examinee placed on a support table positioned for imaging by a medical imaging device;
comparing a level of the detected motion with a predetermined threshold value; and
providing guidance information enabling reducing impairment of image quality due to examinee physical motion, in response to the comparison.

2. The method of claim 1, wherein the guidance information comprises information regarding the level of the detected motion.

3. The method of claim 2, wherein the guidance information further comprises information regarding the threshold value.

4. The method of claim 1, including providing the guidance information by using at least one of text data, graphical data, and audio data.

5. The method of claim 1, wherein, if the level of the detected motion is equal to or greater than the threshold value, the guidance information comprises information indicating that recapture of an image of the examinee is necessary.

6. The method of claim 1, wherein the guidance information comprises information concerning at least one of an angle, a position, and a direction for moving an anatomical part of the examinee.

7. The method of claim 1, further comprising: capturing an image of an imaging part of the examinee,
including providing the image of the imaging part and the guidance information.

8. The method of claim 1, including providing a previously stored exemplary image corresponding to an imaging part of the examinee and the guidance information.

9. The method of claim 1, including providing the guidance information on an in-bore display.

10. The method of claim 1, including capturing an image of an imaging part of the examinee, and detecting the motion of the examinee from the image of the imaging part.

11. The method of claim 10, wherein the capturing comprises: capturing the image of the imaging part by using at least one of a high-speed camera and a wide viewing angle camera.

12. The method of claim 1, wherein the detecting comprises: detecting the motion by using a sensor provided in at least one of the table on which the examinee is placed and a coil mounted on the examinee.

13. The method of claim 12, wherein the sensor comprises at least one of a pressure sensor, a tilt sensor, an acceleration sensor, a gyro sensor, a magnetic field sensor, and an optical sensor.

14. The method of claim 1, further comprising: receiving an identification signal from an identification device mounted on the examinee,
wherein the detecting comprises: detecting the motion based on the identification signal.

15. The method of claim 14, wherein the detecting comprises: detecting the motion based on a change in an intensity of the identification signal.

16. The method of claim 1, further comprising: obtaining medical images using an imaging protocol used to diagnose the examinee,
wherein the detecting comprises: detecting the motion by comparing the medical images.

17. The method of claim 16, wherein the detecting comprises: detecting the motion by comparing image characteristic values of the medical images.

18. The method of claim 1, further comprising: if the level of the detected motion is equal to or greater than the threshold value, suspending an imaging protocol used to diagnose the examinee.

19. The method of claim 18, further comprising: if the level of the detected motion is smaller than the threshold value, resuming the suspended imaging protocol.

20. The method of claim 1, further comprising: if the level of the detected motion is equal to or greater than the threshold value, delaying a resumption of the imaging protocol used to diagnose the examinee.

21. The method of claim 20, further comprising: if the level of the detected motion is smaller than the threshold value, resuming a suspended imaging protocol.

22. The method of claim 1, wherein the medical imaging device comprises a magnetic resonance imaging (MRI) apparatus or a computed tomography (CT) apparatus.

23. The method of claim 1, including providing at least one of a user of the medical imaging apparatus and the examinee with the guidance information.

24. A medical imaging apparatus comprising:
a sensor for detecting motion of an examinee placed on a support table positioned for medical imaging;
a motion calculation unit for comparing a level of the detected motion with a predetermined threshold value; and
an output unit for providing guidance information that is used to guide the examinee relating to the motion in response to the comparison.

25. The medical imaging apparatus of claim 24, wherein the guidance information comprises information concerning the level of the detected motion.

26. The medical imaging apparatus of claim 25, wherein the guidance information further comprises information regarding the threshold value.

27. The medical imaging apparatus of claim 24, wherein the output unit provides the guidance information by using at least one of text data, graphical data, and audio data.

28. The medical imaging apparatus of claim 24, wherein, if the level of the detected motion is equal to or greater than the threshold value, the guidance information comprises information indicating that recapture of an image of the examinee is necessary.

29. The medical imaging apparatus of claim 24, wherein the guidance information comprises information concerning at least one of an angle, a position, and a direction for moving an anatomical part of the examinee.

30. The medical imaging apparatus of claim 24, further comprising: a capturing unit for capturing an image of an anatomical part of the examinee,
wherein the output unit provides the image of the anatomical part and the guidance information.

31. The medical imaging apparatus of claim 24, wherein the output unit provides a previously stored exemplary image corresponding to an anatomical part of the examinee and the guidance information.

32. The medical imaging apparatus of claim 24, wherein the output unit provides the guidance information on an in-bore display.

33. The medical imaging apparatus of claim 24, further comprising: a capturing unit for capturing an image of an anatomical part of the examinee,
wherein the sensor detects the motion of the examinee from an image captured by the capturing unit.

34. The medical imaging apparatus of claim 33, wherein the capturing unit captures the image of the imaging part by using at least one of a high-speed camera and a wide viewing angle camera.

35. The medical imaging apparatus of claim 24, wherein the sensor detects the motion by using a sensor provided in at least one of the table on which the examinee is placed and a coil mounted on the examinee.

36. The medical imaging apparatus of claim 35, wherein the sensor comprises at least one of a pressure sensor, a tilt sensor, an acceleration sensor, a gyro sensor, a magnetic field sensor, and an optical sensor.

37. The medical imaging apparatus of claim 24, wherein the sensor receives an identification signal from an identification device mounted on the examinee, and detects the motion based on the identification signal.

38. The medical imaging apparatus of claim 37, wherein the sensor detects the motion based on a change in an intensity of the identification signal.

39. The medical imaging apparatus of claim 24, further comprising: an image processor for obtaining medical images through an imaging protocol used to diagnose the examinee,
wherein the sensor detects the motion by comparing the medical images.

40. The medical imaging apparatus of claim 38, wherein the sensor detects the motion by comparing image characteristic values of the medical images.

41. The medical imaging apparatus of claim 24, further comprising: an imaging protocol manager for proceeding with a imaging protocol used to diagnose the examinee,
wherein, if the level of the detected motion is equal to or greater than the threshold value, the imaging protocol manager suspends the imaging protocol used to diagnose the examinee.

42. The medical imaging apparatus of claim 41, wherein, if the level of the detected motion is smaller than the threshold value, the imaging protocol manager resumes a suspended imaging protocol.

43. The medical imaging apparatus of claim 24, further comprising:
an imaging protocol manager for proceeding with an imaging protocol used to diagnose the examinee,
wherein, if the level of the detected motion is equal to or greater than the threshold value, the imaging protocol manager holds off a resumption of the imaging protocol used to diagnose the examinee.

44. The medical imaging apparatus of claim 43, wherein, if the level of the detected motion is smaller than the threshold value, the imaging protocol manager resumes the held off imaging protocol.

45. The medical imaging apparatus of claim 24, wherein the medical imaging apparatus comprises an MRI apparatus or a CT apparatus.

46. The medical imaging apparatus of claim 24, wherein the output unit provides at least one of a user of the medical imaging apparatus and the examinee with the guidance information.

47. A non-transitory computer-readable recording medium having recorded thereon a program for executing the method of claim 1.
